(19) United States
(12) Patent Application Publication
(10) Pub. No.: US 2025/0316359 A1
(43) Pub. Date: Oct. 9, 2025

(54) SELECTION OF INTRAOCULAR LENS POWER BASED ON INTEGRATING FINITE ELEMENT MODELING WITH MACHINE LEARNING

(71) Applicant: ALCON INC., Fribourg (CH)

(72) Inventors: Kamal K. Das, Arlington, TX (US); William Jacob Spenner Dolla, Plano, TX (US); Bryan Stanfill, Mansfield, TX (US); Paul J. Missel, Arlington, TX (US); Shruti Siva Kumar, Foothill Ranch, CA (US)

(21) Appl. No.: 19/096,852

(22) Filed: Apr. 1, 2025

Related U.S. Application Data
(60) Provisional application No. 63/631,565, filed on Apr. 9, 2024.

(51) Int. Cl. G16H 20/40; A61B 34/10; A61F 2/16

(52) U.S. Cl. CPC G16H 20/40; A61B 34/10; A61B 2034/104; A61B 2034/105; A61B 2034/108; A61F 2/16

(57) ABSTRACT

A system for selecting an intraocular lens for implantation into an eye includes a controller adapted to selectively execute a finite element model and a machine learning module. The controller is adapted to receive input data, including one or more biometric parameters of the eye. A plurality of capsule parameters are extracted based on the input data, via the machine learning module. The controller is adapted to determine an axial displacement factor based in part on the plurality of capsule parameters, via the finite element model. The axial displacement factor accounts for a predicted axial shift of the intraocular lens after implantation into the eye. The axial displacement factor may be incorporated into the final intraocular lens power when calculating a lens constant parameter utilizing the finite element model and machine learning modules.

### INTRODUCTION

The disclosure relates generally to selection of an intraocular lens for implantation in an eye. More particularly, the disclosure relates to selection of an intraocular lens based on post-implantation axial shift using an integrated finite element modeling and machine learning module. The human lens is generally transparent such that light may travel through it with ease. However, many factors may cause areas in the lens to become cloudy and dense, and thus negatively impact vision quality. The situation may be remedied via a cataract procedure, whereby an artificial lens is selected for implantation into a patient's eye. Indeed, cataract surgery is a common surgery performed all around the world. An important driver of clinical outcome for cataract surgery is the selection of an appropriate intraocular lens power for the best refractive outcomes. Currently, there are several calculators that use various types of pre-operative information pertaining to the patient's eye to select or determine the lens power to be implanted. While remarkable progress has been made in the area of power calculations, challenges remain for eyes falling outside of average biometric parameters.

### SUMMARY

Disclosed herein is a system for selecting an intraocular lens power for implantation into an eye. The system includes a controller having one or more processors and tangible, non-transitory memory on which instructions are recorded. The controller is adapted to selectively execute a finite element model and a machine learning module. The controller is adapted to receive input data, including one or more biometric parameters of the eye, which may be stored as tabular data and/or images. A plurality of capsule parameters corresponding to a lens capsule of the eye are extracted from the input data, via the machine learning module. The controller is adapted to determine an axial displacement factor based in part on the plurality of capsule parameters, via the finite element model. The axial displacement factor accounts for a predicted axial shift of the intraocular lens after implantation into the eye. The controller is adapted to utilize one or more lens constants formulae to recommend an intraocular lens power based on the axial displacement factor.

In some embodiments, the finite element model is tensor-based. The controller may be adapted to employ multi-physics software to execute the finite element model. The controller may be adapted to select the intraocular lens based on the recommended intraocular lens power.

In some embodiments, the plurality of capsule parameters includes a capsule diameter, and a capsule thickness. The plurality of capsule parameters may further include a capsule skew factor that is based on the capsule thickness. For example, the capsule skew factor may be determined as the magnitude of the ratio of the distance from an equatorial plane of the capsule profile in a Y-direction of a centroid of the capsule profile divided by the capsule thickness, the capsule profile being the cross-sectional profile of the lens capsule sliced through the anterior pole and the posterior pole of the lens capsule. The capsule skew factor may be a positive number between 0 and 0.4. The capsule skew factor is zero when the Y-position (e.g., Y-coordinate) of the equatorial plane is exactly halfway between the anterior pole and the posterior pole. The capsule skew factor is greater than zero when the Y-coordinate of the equatorial plane is not halfway between the anterior pole and the posterior pole, which indicates that the respective mass of the lens (as encased by the lens capsule) is relatively greater on the posterior side of the equatorial plane.

Disclosed herein is a method of selecting an intraocular lens for implantation into an eye with a system having a controller with at least one processor and at least one non-transitory, tangible memory. The method includes selectively executing a finite element model and a machine learning module, via the controller. The method includes receiving input data, including one or more biometric parameters of the eye, via the controller. The method includes extracting a plurality of capsule parameters corresponding to a lens capsule of the eye based on the input data, via execution of the machine learning module. The method includes determining an axial displacement factor based in part on the plurality of capsule parameters, via execution of the finite element model. The axial displacement factor accounts for a predicted axial shift of the intraocular lens after implantation into the eye. The method includes utilizing one or more lens constants formulae to recommend an intraocular lens power based on the axial displacement factor.

Disclosed herein is a system for selecting an intraocular lens for implantation into an eye. The system includes a controller having one or more processors and tangible, non-transitory memory on which instructions are recorded. The controller is configured to selectively execute a finite element model and a machine learning module via execution of the instructions by the one or more processors. For example, execution of the instructions by the one or more processors causes the controller to receive input data, including one or more biometric parameters of the eye, and extract a plurality of capsule parameters corresponding to a lens capsule of the eye based on the input data, via the machine learning module. The controller is adapted to determine an axial displacement factor based in part on the plurality of capsule parameters, via the finite element model. The controller is adapted to adjust a lens power of the intraocular lens based in part on the axial displacement factor. The axial displacement factor is a power correction feature accounting for a predicted axial shift of the intraocular lens after implantation into the eye.

The above features and advantages and other features and advantages of the present disclosure are readily apparent from the following detailed description of the best modes for carrying out the disclosure when taken in connection with the accompanying drawings.

### BRIEF DESCRIPTION OF THE DRAWINGS

FIG. 1 is a schematic illustration of a system for selecting an intraocular lens for implantation into an eye, the system having a controller;

FIG. 2 is a schematic sectional view of an example eye;

FIGS. 3A and 3B are schematic flowcharts for respective methods executable by the controller of FIG. 1, in accordance with a first embodiment and a second embodiment;

FIG. 4 is a schematic diagram illustrating an intraocular lens implanted into a lens capsule;

FIG. 5 is a schematic diagram illustrating a set of capsule profiles having the same thickness and diameter aligned at their equatorial positions;

FIG. 6 is a schematic diagram illustrating another set of capsule profiles;

FIG. 7 is a schematic example graph illustrating axial displacement of the intraocular lens on the vertical axis and lens diameter on the horizontal axis;

FIG. 8 is a schematic example illustration of how a machine learning module may be used in place of the finite element analysis module; and

FIG. 9 is a schematic example graph illustrating post-operative spherical equivalent power on the vertical axis and lens power on the horizontal axis.

Representative embodiments of this disclosure are shown by way of non-limiting example in the drawings and are described in additional detail below. It should be understood, however, that the novel aspects of this disclosure are not limited to the particular forms illustrated in the above-enumerated drawings. Rather, the disclosure is to cover modifications, equivalents, combinations, sub-combinations, permutations, groupings, and alternatives falling within the scope of this disclosure as encompassed, for instance, by the appended claims.

### DETAILED DESCRIPTION

Prior to cataract surgery, ophthalmic surgeons make use of a wide variety of algorithms to plan for intraocular lens replacement in order to best correct vision. Power calculation formulae generally assume that the effective lens position of the intraocular lens 12 aligns approximately with the pre-operative capsular bag equator. However, the shape and size of the capsular bag varies within the patient population, making achieving the best refractive outcomes for non-average sized, such as long, short and/or myopic eyes, challenging. Additionally, after implantation, the implanted intraocular lens may move either anteriorly or posteriorly along an axial direction based on haptic and mechanical features of the lens, thus impacting the refractive outcome. One or more embodiments of the present disclosure may utilize one or combinations of finite element modeling and machine learning to facilitate selecting an intraocular lens for implantation.

The drawings are now referred to help further discuss the embodiments of the present disclosure. The figures of the drawings are meant to help ease explanation and are not necessarily meant to be completely accurate. For example, the drawings are not necessarily drawn to scale. Further, although the drawings relate to anatomical components, such components are only meant to be representative and not necessarily completely anatomically correct.

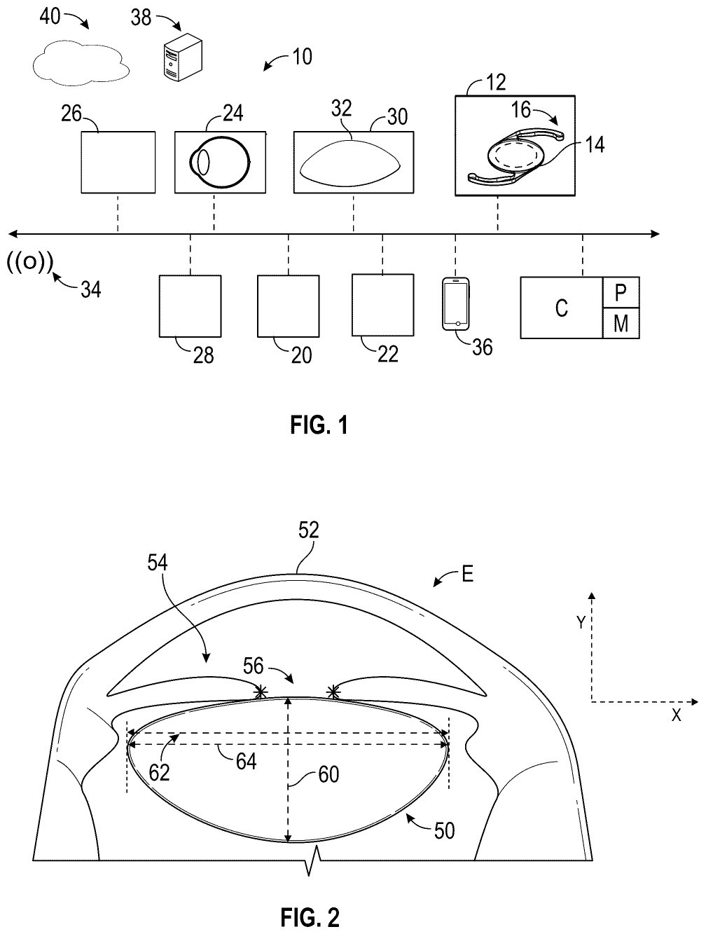

Referring to the drawings, wherein like reference numbers refer to like components, FIG. 1 schematically illustrates a system 10 for selecting an intraocular lens for implantation into an eye E (an example of the eye E is illustrated in FIG. 2). It is to be understood that the intraocular lens 12 may take many different forms and include multiple and/or alternate components. In the embodiment shown in FIG. 1, the intraocular lens 12 includes an optic zone 14 contiguous with supporting structures 16, which are configured to support positioning and retention of the intraocular lens 12.

Referring to FIG. 1, a controller C is adapted to selectively execute a machine learning module 20 and a finite element model 22. The controller C may be configured to communicate with various entities, such as one or more imaging devices 24, and a user interface 26. The imaging devices 24 may include an optical coherence tomography device, a digital or analog microscope, a camera system (e.g., capturing one dimensional or three-dimensional images or videos), an ultrasound machine, a magnetic resonance imaging machine or other imaging device available to those skilled in the art. Additionally, the controller C may be in communication with a database 28 storing input data pertaining to the eye. The controller C is adapted to receive input data, including one or more biometric parameters of the eye E. The biometric parameters may be in tabular or photographic form in some examples. A plurality of capsule parameters 30 related to a lens capsule 32 of the eye E (also indicated in more detail as lens capsule 50 in FIG. 2) are extracted based on the input data, via the machine learning module 20.

The controller C is adapted to determine an axial displacement factor based in part on the plurality of capsule parameters 30, via the finite element model 22. The finite element model 22 may be configured to study the interaction of the intraocular lens 12 with the lens capsule 50 (and/or the lens capsule 32) having various sizes and shapes. The finite element model 22 may also be replaced by a machine learning module trained to replicate the output of the finite element model 22 given the same inputs. This may be useful, for example, in instances in which the finite element model 22 is too time-consuming or computationally expensive. The axial displacement factor accounts for an expected axial shift of the intraocular lens 12 after implantation into the eye E.

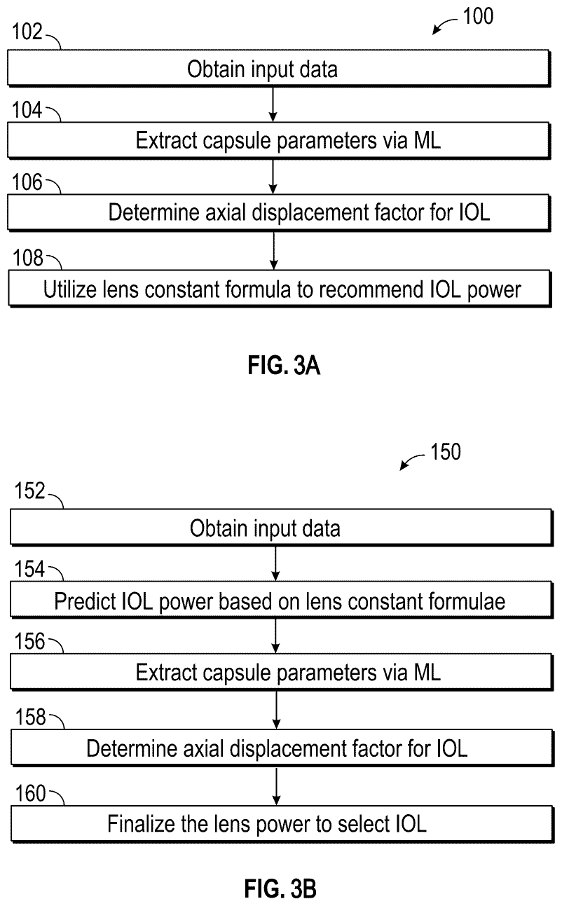

The system 10 provides for a power calculator for the intraocular lens 12 and an adjustment to the recommended power for the intraocular lens 12 that accounts for various capsular bag sizes and shapes and/or other biometric parameters. As described below, the intraocular lens power that accounts for axial displacement may be recommended in various ways. Referring to FIG. 1, the system 10 includes a controller C having at least one processor P and at least one memory M (or non-transitory, tangible computer readable storage medium) on which are recorded instructions for executing a first method 100 and/or a second method 150. Methods 100, 150 are respectively shown in and described below with reference to FIGS. 3A and 3B.

The first method 100 is applicable for IOL power formulae that require a lens constant. In this approach, the axial displacement is accounted for by adjusting the lens constant formulae used to recommend the implant power. In the second method 150, the axial displacement factor is combined with a base power calculation. Here, the power of the intraocular lens 12 is selected based in part on a base IOL (intraocular lens) power calculation and the axial displacement factor. An additional machine learning module may be utilized to combine the axial displacement factor and the base power calculation to recommend the final IOL power. Thus, the system 10 optimizes, improves, or enhances selection of the intraocular lens 12 for a large patient population.

Referring now to FIG. 3A, a flow chart of method 100 executable by the controller C of FIG. 1 is shown. Method 100 need not be applied in the specific order recited herein and some blocks may be omitted. The memory M can store controller-executable instruction sets, and the processor P can execute the controller-executable instruction sets stored in the memory M.

Per block 102 of FIG. 3A, the controller C is configured to receive input data, including one or more biometric parameters of the eye E. The biometric parameters may include an anterior chamber depth, a ciliary process diameter, a sulcus-to-sulcus diameter, corneal power expressed as keratometry values etc. The biometric parameters may be derived from pre-operative images of the eye E. Additionally or alternatively, a surgeon, technician, or other health professional may manually input one or more of the biometric parameters of the eye E.

Per block 104 of FIG. 3A, the controller C is configured to extract a plurality of capsule parameters based on the input data, via the machine learning module 20. An example cross-sectional image of an eye E is shown in FIG. 2 to help illustrate the capsule parameters. The eye E includes a lens capsule 50, cornea 52, iris 54, and pupil 56, with the eye E oriented such that the cornea 52 is at the top of the illustration. The lens capsule 50 may be a thin elastic membrane that surrounds a lens of the eye E such that the lens is encased in the lens capsule 50. In the present disclosure, reference to dimensions corresponding to the lens capsule 50 may refer to dimensions of the space occupied by the lens capsule 50 and not necessarily of the membrane of the lens capsule 50 itself. As such, reference to the dimensions corresponding to the lens capsule 50 may also generally correspond to dimensions of the lens encapsulated by the lens capsule 50.

The lens capsule 50 has a capsule thickness 60 (e.g., the depth of the entire lens capsule 50 from the anterior end to the posterior end of the lens capsule 50), a capsule diameter 62 (e.g., which may refer to the largest width of the lens capsule 50 in a direction orthogonal to the capsule thickness 60), and/or a capsule wall thickness, among other parameters. The capsule parameters can include the capsule thickness 60, the capsule diameter 62, the capsule wall thickness, or other parameters. In some embodiments, the plurality of capsule parameters can include a capsule skew factor, which captures asymmetry in the lens capsule 50 as referenced using an equatorial plane 64 of the lens capsule 50. The equatorial plane 64 may be a plane that intersects the lens capsule 50 at the location of the lens capsule 50 that corresponds to the capsule diameter 62 such that the equatorial plane 64 may be disposed at the widest portion of the lens capsule 50. Stated differently, the equatorial plane 64 may be the plane defined by the line connecting the points of intersection between the curve defining the anterior of the lens capsule 50 with the posterior curve and extending perpendicular to the page. In the illustrated example of FIG. 2, the cross-sectional image may be referenced using a horizontal X-axis, a vertical Y-axis, and a corresponding Z-axis that is orthogonal to the X and Y axes (e.g., that passes through the page). Using this reference system, the equatorial plane 64 may accordingly be an XZ plane disposed at a particular location along the Y-axis.

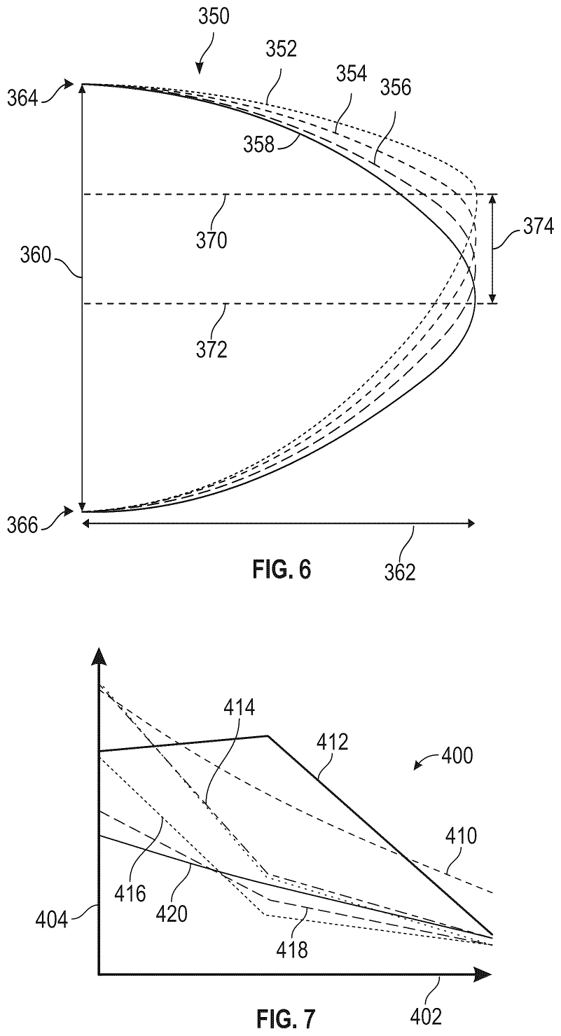

In these and other embodiments, and as described in further detail below with respect to FIG. 5 and/or FIG. 6, in some embodiments the skewness may be indicated as a skew factor. As indicated above, the skew factor may be based on a positional relationship between a location of a centroid of the lens capsule 50 (e.g., the geometric center of the lens capsule 50, which may also be the center of mass within the lens capsule 50 assuming uniform mass density) in relation to the equatorial plane 64. For example, the skew factor may be determined using the location of the centroid (also referred to as “centroidal location”) along the vertical axis (e.g., illustrated Y-axis) in relation to the vertical axis (or “Y-axis”) location of the equatorial plane 64. Using this determination technique, the skew factor is a positive number that may be between 0 and 0.4 and that may typically fall between 0 and 0.2.

The machine learning module 20 may include any suitable artificial intelligence model. For example, the machine learning module 20 may include one or more neural networks or other machine learning algorithms trained to extract the capsule parameters from the biometric data included in the input data. For instance, the machine learning module 20 may be configured to process raw biometric imaging data, such as optical coherence tomography scans, ultrasound images, and/or other ocular imaging modalities.

To extract the plurality of capsule parameters, the machine learning module 20 may first preprocess the input biometric data, for example by applying image enhancement techniques, segmentation algorithms, or feature detection methods. The preprocessed data may then be input into one or more trained neural networks. These neural networks may be convolutional neural networks or other architectures suited for image analysis and feature extraction.

The neural networks may be trained on a large dataset of labeled biometric images to recognize and measure key anatomical features of the lens capsule 50. For example, the neural networks may be trained to identify the anterior and posterior poles of the lens capsule 50, measure the capsule diameter 62 and/or capsule thickness 60, and/or determine the centroid location.

Based on the extracted features, the machine learning module 20 may calculate derived capsule parameters such as the capsule skew factor. In these and other embodiments, the machine learning module 20 may output a set of quantitative capsule parameters including the capsule diameter, capsule thickness, capsule wall thickness, capsule skew factor, and other relevant parameters.

Per block 106 of FIG. 3A, the controller C is adapted to determine an axial displacement factor based in part on the plurality of capsule parameters, via the finite element model 22. The axial displacement factor may account for the predicted axial shift of the intraocular lens 12 after implantation into the eye E. In these and other embodiments, the axial displacement factor may represent how much the intraocular lens 12 is expected to move along the optical axis of the eye E following implantation. This predicted movement may be influenced by the capsule parameters. By incorporating this axial displacement prediction into the lens power calculation, the system may be able to recommend an intraocular lens power that accounts for the expected post-operative position of the lens. This may allow for more accurate refractive outcomes by adjusting the lens power to compensate for the predicted axial shift after implantation.

In some embodiments, due to the computation complexity of finite element models, a machine learning module (e.g., the machine learning module 20) may be trained to emulate the finite element model 22 such that the machine learning module may be used as a proxy for the finite element model 22. Therefore, in the present disclosure reference to a “finite element model” may also refer to a machine learning module that has been configured to (e.g., trained to) emulate a finite element model.

In some embodiments, the controller C is adapted to predict the effective lens position of the intraocular lens 12 by incorporating the capsule parameters which may or may not be extracted from the biometric parameters (e.g., through the machine learning module 20). Additionally or alternatively, the finite element model 22 may be configured to predict the effective lens position of the intraocular lens 12, relative to the anterior or posterior surface of the cornea, taking into account the settling of the IOL haptics in the equatorial position of the capsule and the subsequent axial displacement, both of which may be influenced by the capsule parameters. The plurality of capsule parameters is fed into the finite element model 22 to estimate axial displacement of the intraocular lens 12.

For example, in general the finite element model 22 simulates the physical system using a numerical technique called the finite element method. In some embodiments, the finite element model is tensor-based. As understood by those skilled in the art, tensor-based models employ tensor mathematics to represent the properties and behavior of a physical system. Tensors are mathematical objects that generalize scalars, vectors, and matrices. Tensor variables such as stress and strain may be represented at integration points within each element in finite element analysis, capturing directional dependencies. The controller C may employ multi-physics software to execute the finite element model 22. As understood by those skilled in the art, multi-physics simulation software refers to software that can simulate models from different physical domains (e.g., interacting physical models), such as the COMSOL multi-physics software. In some embodiments, due to the computation complexity of finite element models, the machine learning module 20 may be trained to emulate the finite element model 22.

As a more specific example for the present disclosure, the finite element model 22 may include a computational model that divides the lens capsule 50 and intraocular lens 12 into small discrete elements. These elements may be interconnected to form a mesh representation of a capsule-IOL system. The finite element model 22 may incorporate material properties of the lens capsule 50 and intraocular lens 12, as well as boundary conditions and loading scenarios to simulate the post-implantation behavior of the intraocular lens 12.

To determine the axial displacement factor, the finite element model 22 may take the extracted capsule parameters as inputs. These parameters may include the capsule diameter, thickness, wall thickness, and skew factor. The finite element model 22 may then simulate the interaction between the intraocular lens 12 and lens capsule 50 under physiological conditions as indicated by the capsule parameters. This simulation may account for factors such as capsule elasticity, IOL haptic compression, and gravitational effects.

The finite element analysis performed by the finite element model 22 may solve for the deformation and stress distribution in the capsule-IOL system. From this solution, the axial displacement of the intraocular lens 12 relative to its initial position may be calculated. This displacement value may serve as the axial displacement factor used to adjust IOL power calculations. As mentioned above, in some embodiments, the displacement value may be relative to the position of the equatorial plane 64 of the lens capsule 50.

FIG. 7 is a schematic example graph showing a set 400 of lens capsule traces, obtained via the finite element model 22. The vertical axis 404 may correspond to the Y-axis of FIG. 2 and indicates axial displacement and the horizontal axis 402 may correspond to the X-axis of FIG. 2 and indicates capsule diameter. The set 400 includes traces 410, 412, 414, 416, 418, and 420. Traces 410, 412, 414, 416, 418, and 420 respectively correspond to a capsule thickness (in mm) and capsule skew factor as follows: [3.8 mm, 0.11], [3.8 mm, 0.14], [4.8 mm, 0.11], [4.3 mm, 0.07], [4.3 mm, 0.11], and [4.8 mm, 0.14]. FIG. 7 shows the variability in axial posterior displacement of the intraocular lens (relative to the equatorial plane position) with capsule size and shape.

In some embodiments, the finite element model 22 is adapted to employ trial data to create a capsule finite element seed database that covers the human physiological ranges of lens diameter, lens thickness, and effective lens position for a target intraocular lens. The trial data may be stored in the database 28. The interaction and movement of the intraocular lens 12 after implantation varies with capsule shape and size, thereby influencing the effective lens position of the implanted intraocular lens 12. The finite element model 22 may be expanded to cover each of the capsule size combinations observed clinically. For example, the database 28 may be updated with clinical observations or other data over time such that the finite element model 22 may include additional traces or observations. In these and other embodiments, the trial data may then be used to train machine learning algorithms to rapidly predict displacement for new patient-specific capsule parameters without needing to re-run the full finite element simulation each time.

Per block 108 of FIG. 3A, the method 100 includes utilizing one or more lens constant formulae to recommend the implant power based on the axial displacement factor (per block 106) that was learned from the finite element model 22 and/or machine learning module 20 for various eye types or capsules. In other words, the axial displacement factor may be used to adjust the lens constant that goes into the power calculator. Any suitable lens constant formulae available to those skilled in the art may be employed.

Referring now to FIG. 3B, a flow chart of method 150 executable by the controller C of FIG. 1 is shown. Method 150 need not be applied in the specific order recited herein and some blocks may be omitted. Per block 152 of FIG. 3B, the controller C is configured to obtain input data, including one or more biometric parameters of the eye E. The biometric parameters may include an anterior chamber depth, a ciliary process diameter, a sulcus-to-sulcus diameter, corneal power expressed as keratometry values etc. The biometric parameters may be derived from pre-operative images of the eye E.

Per block 154 of FIG. 3B, the controller C is adapted to perform a base IOL (intraocular lens) power calculation for obtaining a base optical power of an intraocular lens 12. IOL power calculators include but are not limited to the Barrett Universal formula, the Kane formula, etc. The controller C may input the biometric parameters (from block 102) into an optical design software that uses an optical eye model available to those skilled in the art. The controller C may adopt ray tracing techniques tracing the propagation of light through the optical eye model to predict IOL power based on one or more lens constants. The base IOL power is selected as the one which focuses light rays directly onto the retina of the eye E, minimizing or reducing spherical and other optical aberrations.

Per block 156 of FIG. 3B, the controller C is configured to extract a plurality of capsule parameters based on the input data, via the machine learning module 20 (e.g., such as described above). The plurality of capsule parameters includes the capsule thickness 60 and a capsule diameter 62, shown in FIG. 2. The plurality of capsule parameters includes a capsule skew factor, which captures asymmetry in the lens capsule relative to an equatorial plane 64.

Per block 158 of FIG. 3B, the method 150 includes determining an axial displacement factor for the intraocular lens 12. In some embodiments, the determining of the axial displacement factor may include determining an expected axial displacement of the intraocular lens 12 based in part on the plurality of capsule parameters, through execution of the finite element model 22, such as described above.

In these and other embodiments, the method 150 may include determining (e.g., calculating) an adjustment factor for the power of the intraocular lens 12 based on the axial displacement factor. Additionally or alternatively, the adjustment factor may be determined based on other factors that may be included in or based on the biometry and/or size of the capsule that may be determined based on the achieved knowledge included in the capsule parameters. The adjustment factor may indicate an amount of adjustment that may be made to the lens power.

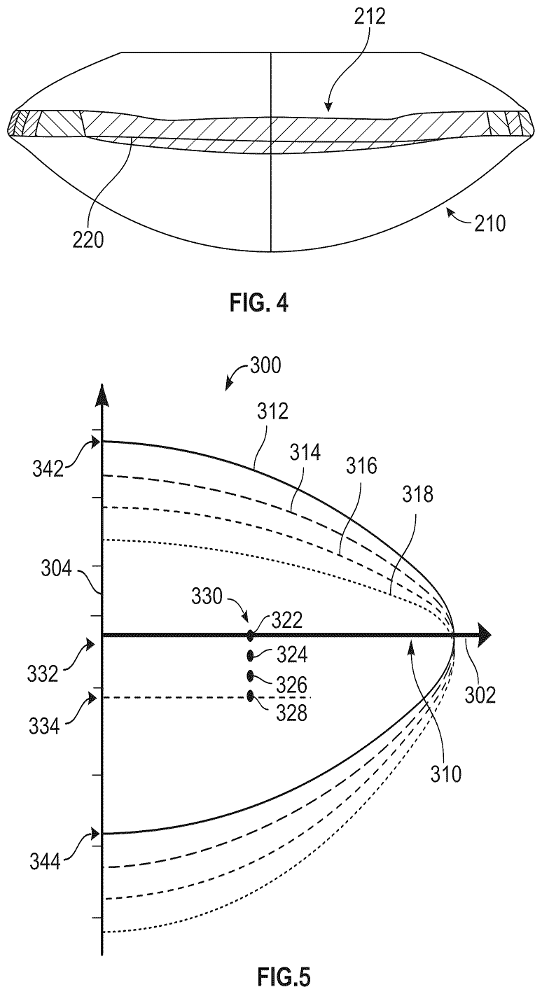

FIG. 4 is a schematic diagram illustrating an intraocular lens 212 implanted into a lens capsule 210, based on the finite element model 22. As shown in FIG. 4, the intraocular lens 212 does not align with or is not symmetric relative to an equatorial plane 220 of the lens capsule 210. Note that the line referencing the equatorial plane 220 is not necessarily a true representation of the equator of the lens capsule 210 due to the intraocular lens 212 skewing the equatorial plane to some extent. In the embodiment shown in FIG. 4, the capsule skew factor is biased towards the posterior pole (above zero). Or stated another way, because more of the intraocular lens 212 is disposed towards the anterior region of the capsule 210, the capsule skew factor is biased towards the posterior pole. In the illustrated example, the posterior direction is downward and the anterior direction is upward in FIG. 4. The finite element model 22 may be replaced with a machine learning module to speed up computational time and reduce computational demands, such as described above.

Returning to FIG. 3B, per block 160 of FIG. 3B, the method 150 includes finalizing the lens power for the intraocular lens 12 based on the axial displacement factor and/or the lens power adjustment factor. In these and other embodiments, finalizing the lens power may include adjusting the base IOL power is based on the axial displacement factor. Additionally or alternatively, the intraocular lens 12 is selected based in part on the adjusted base IOL power. An additional machine learning model may be used to combine the axial displacement factor and base IOL power in some embodiments.

FIG. 5 illustrates a set of capsule profiles 300 that are aligned at an equatorial plane 310. Stated another way, the equatorial planes of the different capsule profiles 300 are aligned such that the equatorial plane 310 represents the equatorial planes of all of the capsule profiles. The capsule profiles 312, 314, 316, 318 are shown for lenses having the same capsule thickness (along the vertical axis (or also referred to as the Y-axis) 304) and the same capsule diameter (along the horizontal axis (or also referred to as the X-axis) 302), but for four different values of the capsule skew factor. The capsule profiles 312, 314, 316, 318 respectively define a respective centroid 322, 324, 326, and 328. As indicated elsewhere in the present disclosure, the centroid may generally understood to be the center of mass of a geometric object of uniform density. Each of the respective centroids 322, 324, 326, and 328 defines a respective X-coordinate 330 and a respective Y-coordinate 332 (e.g., Y-coordinate 334 of the centroid 328). The lens capsules define respective capsule profiles (e.g., the capsule profiles 312, 314, 316, 318), which are cross-sectional profiles of different lens capsules sliced through respective anterior and posterior poles of the different lens capsules. For example, FIG. 5 illustrates an example relative location of an anterior pole 342 and an example relative location of a posterior pole 344 for the capsule profile 312. Additionally or alternatively, the equatorial plane 310 may be halfway between the anterior pole 342 and the posterior pole 344 of the capsule profile 312 such that the equatorial plane 310 of the capsule profile 312 may be at the center of the thickness of the capsule profile 312. Note that the other equatorial planes 310 of the other capsule profiles (e.g., the capsule profiles 314, 316, and/or 318) may not be located halfway between their respective anterior and posterior poles, such that their respective equatorial planes 310 are not at the center of the capsule thickness of such lens capsules. It is understood that the FIGS. are not drawn to scale.

In some embodiments, the capsule skew factor may be defined as the ratio of the distance of the centroid (e.g., centroid 322) of the corresponding capsule provide (e.g., the capsule profile 312) in a Y-direction from the equatorial plane divided by the capsule thickness 60 (e.g., the distance between the respective anterior and posterior poles of the corresponding capsule profile). Further, the centroid locations may align with the respective equatorial planes of their respective capsule profiles in instances in which the equatorial planes are disposed halfway between the corresponding anterior and posterior poles. However, the centroid locations may not align with the respective equatorial planes of their respective capsule profiles in instances in which the equatorial planes are not disposed halfway between the corresponding anterior and posterior poles.

The capsule skew factor may accordingly be zero when the Y-axis position of the respective centroids of the respective capsule profiles is exactly halfway between the corresponding anterior pole and posterior pole. For example, the capsule skew factor of the capsule profile 312 may be zero because the equatorial plane 310 and the Y-coordinate 332 of the respective centroid 322 of the capsule profile 312 are exactly halfway between the anterior pole 342 and the posterior pole 344. However, the capsule skew factors of the other capsule profiles 314, 316, and 318 may not be zero because their respective centroids 324, 326, and 328 and the equatorial plane 310 (which as indicated above represents the locations of the corresponding equatorial planes) may not be halfway between their respective anterior and posterior poles.

FIG. 6 is a schematic diagram illustrating another set of capsule profiles 350, including capsule profiles 352, 354, 356, 358. The set of capsule profiles 350 are shown for lens capsules having the same capsule thickness (along the vertical axis (or also referred to as the Y-axis) 360) and the same capsule diameter (along the horizontal axis (or also referred to as the X-axis) 362), but for four different values of the capsule skew factor. In FIG. 6, the capsule profiles 352, 354, 356, 358 are aligned at an anterior pole 364 and posterior pole 366 that correspond to all of the capsule profiles of the set of capsule profiles 350. The capsule profiles 352, 354, 356, 358 define a variably displaced structure, for example, the capsule profiles 352, 358 respectively define equatorial planes 370, 372, that are axially shifted by a distance 374. In one example, the height/thickness of the capsule along the vertical axis 360 is about 5.6 mm, the width (to their respective apices) along the horizontal axis 362 is about 5 mm, and the axial-shift distance 374 is about 1.4 mm.

When the capsule skew factor is zero, the Y-coordinate of the corresponding equatorial plane (and corresponding centroid) is exactly halfway between the anterior pole 364 and the posterior pole 366. For example, the capsule skew factor for the capsule profile 358 may be zero because the Y-coordinate of the equatorial plane 372 corresponding thereto may be zero. As the capsule skew factor increases the corresponding equatorial plane moves toward the anterior pole 364, the centroid is disposed away from the corresponding equatorial plane in the posterior direction, and the mass of the lens capsule (see FIG. 4) on the anterior side of the equatorial plane decreases compared to the posterior side. For example, the skew factor for the capsule profile 352 may be greater than that of the capsule profiles 354, 356, and 358 because the equatorial plane 370 corresponding to the capsule profile 352 may be furthest from the center of the anterior pole 364 and the posterior pole 366 and closest to the anterior pole 364 as compared to the respective equatorial poles of the capsule profiles 354, 356, and 358. As indicated elsewhere, the capsule skew factor may be between 0 and 0.4 and may typically be between 0 and 0.2.

The machine learning module 20 may include a neural network trained using training datasets. The training process occurs in a closed loop or iterative fashion, with the neural network being trained until a certain criteria is met, e.g., until the discrepancy between the network outcome and ground truth reaches a point below a certain threshold. As a predefined loss function related to the training dataset is minimized, the neural network reaches convergence. The convergence signals the completion of the training. The system 10 may be configured to be “adaptive” and updated periodically after the collection of additional training data for the machine learning module 20. It is to be understood that the system 10 is not limited to a specific neural network methodology.

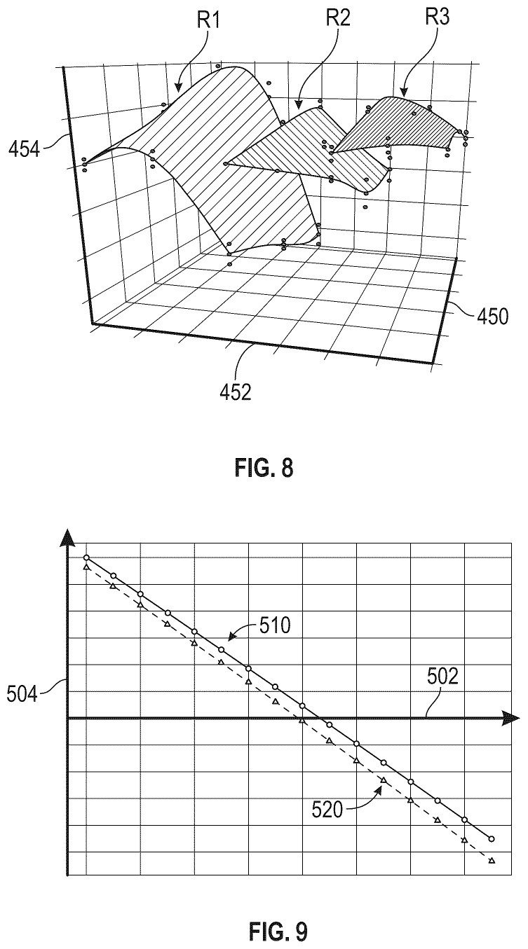

FIG. 8 provides an example of how a machine learning module 20 may be used to emulate the finite element analysis model 22. Referring to FIG. 8, an example graph of various patient data distribution surfaces, including a first data region R1, a second data region R2 and a third data region R3 is shown in three spatial dimensions. The data distribution surfaces are patient-specific finite element models shown along a first axis 450 (capsule thickness in mm), a second axis 452 (capsule diameter in mm), and a third axis 454 (diopter strength or optical power). The points in each region represent the inputs (first axis 450 and second axis 452) and outputs (third axis 454) of the finite element model for a range of capsule skew values. The surfaces represent a machine learning module 20 trained on the finite element model 22 over the same range of data.

FIG. 9 shows a comparison of base IOL power with the corrected or adjusted base IOL power. Line 510 depicts the uncorrected power. Line 520 depicts the adjusted base IOL power. The horizontal axis 502 indicates IOL power (Diopters) while the vertical axis 504 indicates the predicted post-operative IOL spherical equivalent power (Diopter). The adjusted base IOL power may be output to a lens selection module (see FIG. 1) for selecting an intraocular lens 12 for implantation into the eye E.

In summary, the system 10 selectively executes a finite element model 22 with a machine learning module 20, to determine an axial displacement factor for augmenting a base IOL (intraocular lens) power calculation, thereby optimizing, adjusting, or improving refractive outcomes. The system 10 may be expanded to various types of intraocular lenses and their target populations by first generating seed data (of effective lens position) using the finite element model 22. Thereafter, the finite element model 22 may suggest a power correction for the combination of the capsule parameters of each specific patient in a clinical setting, without having to regenerate patient-specific finite element model data.

The various components of the system 10 of FIG. 1 may communicate via a wireless network 34. The network 34 may be a bus implemented in various ways, such as for example, a serial communication bus in the form of a local area network. The local area network may include, but is not limited to, a Controller Area Network (CAN), a Controller Area Network with Flexible Data Rate (CAN-FD), Ethernet, blue tooth, WIFI and other forms of data connection. The network 34 may be a Wireless Local Area Network (LAN) which links multiple devices using a wireless distribution method, a Wireless Metropolitan Area Networks (MAN) which connects several wireless LANs or a Wireless Wide Area Network (WAN). Other types of connections may be employed.

Referring to FIG. 1, the controller C may be configured to receive and transmit data through a mobile application 36, which may be installed on a smartphone, laptop, tablet, desktop or other electronic device and may include a touch screen interface or I/O device such as a keyboard or mouse. The circuitry and components of a mobile application (“apps”) available to those skilled in the art may be employed. The controller C may interact with a cloud unit 40 and/or a remote server 38 and be configured to share data across all clinical sites employing the system 10. In some embodiments, the mobile application 36 is employed to interface with a remote server 38 in the cloud unit 40, upon which the method 100 is executed, and the recommended lens correction factor/feature is fed back to the mobile application 36 running on the processor P. The cloud unit 40 may include one or more servers hosted on the Internet to store, manage, and process data. The remote server 38 may be a private or public source of information maintained by an organization, such as for example, a research institute, a company, a university and/or a hospital.

The controller C of FIG. 1 includes a computer-readable medium (also referred to as a processor-readable medium), including a non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random-access memory (DRAM), which may constitute a main memory. Such instructions may be transmitted by one or more transmission media, including coaxial cables, copper wire and fiber optics, including the wires that comprise a system bus coupled to a processor of a computer. Some forms of computer-readable media include, for example, a floppy disk, a flexible disk, hard disk, magnetic tape, other magnetic medium, a CD-ROM, DVD, other optical medium, a physical medium, a RAM, a PROM, an EPROM, a FLASH-EEPROM, other memory chip or cartridge, or other medium from which a computer can read.

Look-up tables, databases, data repositories or other data stores described herein may include various kinds of mechanisms for storing, accessing, and retrieving various kinds of data, including a hierarchical database, a set of files in a file storage system, an application database in a proprietary format, a relational database management system (RDBMS), etc. Each such data store may be included within a computing device employing a computer operating system such as one of those mentioned above and may be accessed via a network in one or more of a variety of manners. A file system may be accessible from a computer operating system and may include files stored in various formats. An RDBMS may employ the Structured Query Language (SQL) in addition to a language for creating, storing, editing, and executing stored procedures, such as the PL/SQL language mentioned above.

The flowchart shown in the FIGS. illustrates an architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by specific purpose hardware-based systems that perform the specified functions or acts, or combinations of specific purpose hardware and computer instructions. These computer program instructions may also be stored in a computer-readable medium that can direct a controller or other programmable data processing apparatus to function in a particular manner, such that the instructions stored in the computer-readable medium produce an article of manufacture including instructions to implement the function/act specified in the flowchart and/or block diagram blocks.

The numerical values of orders (e.g., of quantities or conditions) in this specification, including the appended claims, are to be understood as being modified in each respective instance by the term “about” whether or not “about” actually appears before the numerical value. “About” indicates that the stated numerical value allows some slight imprecision (with some approach to exactness in the value; about or reasonably close to the value; nearly). If the imprecision provided by “about” is not otherwise understood in the art with this ordinary meaning, then “about” as used herein indicates at least variations that may arise from ordinary methods of measuring and using such orders. In addition, disclosure of ranges includes disclosure of each value and further divided ranges within the entire range. Each value within a range and the endpoints of a range are hereby disclosed as separate embodiments.

The detailed description and the drawings or FIGS. are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings, or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

The subject technology of the present disclosure is illustrated, for example, according to various aspects described below. Various examples of aspects of the present disclosure are described as numbered examples (1, 2, 3, etc.) for convenience. These are provided as examples and do not limit the present disclosure. The aspects of the various implementations described herein may be omitted, substituted for aspects of other implementations, or combined with aspects of other implementations unless context dictates otherwise. For example, one or more aspects of Example 1 below may be omitted, substituted for one or more aspects of another example (e.g., Example 2) or examples, or combined with aspects of another example. The following is a non-limiting summary of some example implementations presented herein.

Example 1. A system for selecting an intraocular lens for implantation into an eye, the system comprising:
a controller having a processor and tangible, non-transitory memory on which instructions are recorded;
wherein the controller is configured to selectively execute a finite element model and a machine learning module, execution of the instructions by the one or more processors cause the controller to:
   obtain input data, including one or more biometric parameters of the eye;
   extract a plurality of capsule parameters corresponding to a lens capsule of the eye based on the input data, via the machine learning module;
   determine an axial displacement factor based in part on the plurality of capsule parameters, via the finite element model, the axial displacement factor accounting for a predicted axial shift of the intraocular lens after implantation into the eye; and
   utilize one or more lens constants formulae to recommend an intraocular lens power based on the axial displacement factor.

Example 2. The system of Example 1, wherein the finite element model is tensor-based, the controller is adapted to employ multi-physics software to execute the finite element model.

Example 3. The system of Example 1 or Example 2, wherein the controller is adapted to select the intraocular lens based on the recommended intraocular lens power.

Example 4. The system of any one of Examples 1-3, wherein the plurality of capsule parameters include a capsule diameter, and a capsule thickness.

Example 5. The system of Example 4, wherein the plurality of capsule parameters include a capsule skew factor that is based on the capsule thickness.

Example 6. The system of Example 5, wherein the capsule skew factor is a ratio of a Y-coordinate of a centroid of a capsule profile divided by the capsule thickness, the capsule profile is a cross-sectional profile of the lens capsule sliced through an anterior pole and a posterior pole of the lens capsule.

Example 7. The system of Example 6, wherein the capsule skew factor is between 0 and 0.2.

Example 8. The system of Example 6, wherein the capsule skew factor is zero when the Y-coordinate of an equatorial plane is exactly halfway between the anterior pole and the posterior pole.

Example 9. The system of Example 8, wherein the capsule skew factor is greater than zero when the Y-coordinate of the equatorial plane is not halfway between the anterior pole and the posterior pole, and a respective mass of a lens of the eye is relatively greater on the anterior posterior side of the equatorial plane.

Example 10. A method of selecting an intraocular lens for implantation into an eye with a system having a controller with at least one processor and at least one non-transitory, tangible memory, the method comprising:
selectively executing a finite element model and a machine learning module, via the controller;
receiving input data, including one or more biometric parameters of the eye, via the controller;
extracting a plurality of capsule parameters corresponding to a lens capsule of the eye based on the input data, via execution of the machine learning module;
determining an axial displacement factor based in part on the plurality of capsule parameters, via execution of the finite element model, the axial displacement factor accounting for a predicted axial shift of the intraocular lens after implantation into the eye; and
utilizing one or more lens constants formulae to recommend an intraocular lens power based on the axial displacement factor.

Example 11. The method of Example 10, further comprising:
selecting the finite element model to be tensor-based, the controller is adapted to employ multi-physics software to execute the finite element model.

Example 12. The method of Example 10 or 11, further comprising:
selecting the machine learning module to be a neural network.

Example 13. The method of any one of Examples 10-12, further comprising:

1selecting the plurality of capsule parameters to include a capsule diameter, and a capsule thickness.

Example 14. The method of Example 13, further comprising:
selecting the plurality of capsule parameters to include a capsule skew factor.

Example 15. The method of Example 14, further comprising:
selecting the capsule skew factor to be a ratio of a Y-coordinate of a centroid of a capsule profile divided by the capsule thickness, the capsule profile is an outer profile of the lens sliced through an anterior pole and a posterior pole of the lens capsule.

Example 16. The method of Example 15, wherein the capsule skew factor is between 0 and 0.2.

Example 17. The method of Example 15, further comprising:
selecting the capsule skew factor to be zero when the Y-coordinate of an equator is exactly halfway between the anterior pole and the posterior pole.

Example 18. The method of Example 17, further comprising:
selecting the capsule skew factor to be greater than zero when the Y-coordinate of the equatorial plane is not halfway between the anterior pole and the posterior pole, and a respective mass of a lens of the eye is relatively greater on the posterior side of the equatorial plane.

Example 19. A system for selecting an intraocular lens for implantation into an eye, the system comprising:
a controller having one or more processors and tangible, non-transitory memory on which instructions are recorded;
wherein the controller is configured to selectively execute a first machine learning module and a second machine learning model trained to emulate a finite element model, execution of the instructions by the one or more processors causes the controller to:
   receive input data, including one or more biometric parameters of the eye;
   extract a plurality of capsule parameters based on the input data, via the first machine learning module;
   determine an axial displacement factor based in part on the plurality of capsule parameters, via the second machine learning model trained to emulate the finite element model; and
   adjust a lens power of the intraocular lens based in part on the axial displacement factor, the axial displacement factor is a power correction feature accounting for a predicted axial shift of the intraocular lens after implantation into the eye.

What is claimed is:

1. A system for selecting an intraocular lens for implantation into an eye, the system comprising:
a controller having one or more processors and tangible, non-transitory memory on which instructions are recorded;
wherein the controller is configured to selectively execute a finite element model and a machine learning module, execution of the instructions by the one or more processors causing the controller to:
obtain input data, including one or more biometric parameters of the eye;
extract a plurality of capsule parameters corresponding to a lens capsule of the eye based on the input data, via the machine learning module;
determine an axial displacement factor based in part on the plurality of capsule parameters, via the finite element model, the axial displacement factor accounting for a predicted axial shift of the intraocular lens after implantation into the eye; and
utilize one or more lens constants formulae to recommend an intraocular lens power based on the axial displacement factor.

2. The system of claim 1, wherein the finite element model is tensor-based, the controller being adapted to employ multi-physics software to execute the finite element model.

3. The system of claim 1, wherein the controller is adapted to select the intraocular lens based on the recommended intraocular lens power.

4. The system of claim 1, wherein the plurality of capsule parameters includes a capsule diameter, and a capsule thickness.

5. The system of claim 4, wherein the plurality of capsule parameters includes a capsule skew factor that is based on the capsule thickness.

6. The system of claim 5, wherein the capsule skew factor is a ratio of a Y-coordinate of a centroid of a capsule profile divided by the capsule thickness, the capsule profile being a cross-sectional profile of the lens capsule sliced through an anterior pole and a posterior pole of the lens capsule.

7. The system of claim 6, wherein the capsule skew factor is between 0 and

0. 2.

8. The system of claim 6, wherein the capsule skew factor is zero when the Y-coordinate of an equatorial plane is exactly halfway between the anterior pole and the posterior pole.

9. The system of claim 8, wherein the capsule skew factor is greater than zero when the Y-coordinate of the equatorial plane is not halfway between the anterior pole and the posterior pole, and a respective mass of a lens of the eye is relatively greater on a posterior side of the equatorial plane.

10. A method of selecting an intraocular lens for implantation into an eye with a system having a controller with at least one processor and at least one non-transitory, tangible memory, the method comprising:
selectively executing a finite element model and a machine learning module, via the controller;
receiving input data, including one or more biometric parameters of the eye, via the controller;
extracting a plurality of capsule parameters corresponding to a lens capsule of the eye based on the input data, via execution of the machine learning module;
determining an axial displacement factor based in part on the plurality of capsule parameters, via execution of the finite element model, the axial displacement factor accounting for a predicted axial shift of the intraocular lens after implantation into the eye; and
utilizing one or more lens constants formulae to recommend an intraocular lens power based on the axial displacement factor.

11. The method of claim 10, further comprising:
selecting the finite element model to be tensor-based, the controller being adapted to employ multi-physics software to execute the finite element model.

12. The method of claim 10, further comprising:
selecting the machine learning module to be a neural network.

13. The method of claim 10, further comprising:
selecting the plurality of capsule parameters to include a capsule diameter, and a capsule thickness.

14. The method of claim 13, further comprising:
selecting the plurality of capsule parameters to include a capsule skew factor.

15. The method of claim 14, further comprising:
selecting the capsule skew factor to be a ratio of a Y-coordinate of a centroid of a capsule profile divided by the capsule thickness, the capsule profile being a cross-sectional profile of the lens capsule sliced through an anterior pole and a posterior pole of the lens capsule.

16. The method of claim 15, wherein the capsule skew factor is between 0 and 0.2.

17. The method of claim 15, further comprising:
selecting the capsule skew factor to be zero when the Y-coordinate of an equatorial plane is exactly halfway between the anterior pole and the posterior pole.

18. The method of claim 17, further comprising:
selecting the capsule skew factor to be greater than zero when the Y-coordinate of the equatorial plane is not halfway between the anterior pole and the posterior pole, and a respective mass of a lens of the eye is relatively greater on a posterior side of the equatorial plane.

19. A system for selecting an intraocular lens for implantation into an eye, the system comprising:
a controller having one or more processors and tangible, non-transitory memory on which instructions are recorded;
wherein the controller is configured to selectively execute a first machine learning module and a second machine learning model trained to emulate a finite element model, execution of the instructions by the one or more processors causing the controller to:
receive input data, including one or more biometric parameters of the eye;
extract a plurality of capsule parameters based on the input data, via the first machine learning module;
determine an axial displacement factor based in part on the plurality of capsule parameters, via the second machine learning model trained to emulate the finite element model; and
adjust a lens power of the intraocular lens based in part on the axial displacement factor, the axial displacement factor being a power correction feature accounting for a predicted axial shift of the intraocular lens after implantation into the eye.
